(19) United States
(12) Patent Application Publication
(10) Pub. No.: US 2019/0274610 A1
(43) Pub. Date: Sep. 12, 2019

(54) Cognitive and Neuropsychic Improvement and Evaluation/Analysis of Alzheimer's Disease and Other Neurological Diseases with Augmented and Mixed Reality

(71) Applicants: Yongbo Wang, Beijing (CN); Zhongsheng Ji, Dublin, CA (US)

(72) Inventors: Yongbo Wang, Beijing (CN); Zhongsheng Ji, Dublin, CA (US)

(21) Appl. No.: 16/211,187

(22) Filed: Dec. 5, 2018

Related U.S. Application Data
(60) Provisional application No. 62/640,858, filed on Mar. 9, 2018.

(51) Int. Cl. A61B 5/16; A61B 5/00

(52) U.S. Cl. CPC A61B 5/165; A61B 5/7445; A61B 5/0048; A61B 5/168; A61B 5/0051; A61B 5/162; A61B 5/4088; A61B 5/7405

(57) ABSTRACT

The present invention provides method for the quantitative or qualitive evaluation and ement of the user's cognitive ability, screening and improving pathological neurological ions. This is achieved by integrating and adapting current-in-practice cognitive assessment niques to an augmented/mixed reality system, which presents signals and stimulations in various forms to the user meanwhile recording user's surroundings, performance, and responses. The stimulations enhance users' cognitive functions, and the acquired data are evaluated by statistic methods to generate an evaluation that is more standardized and reflective of user's actual condition than current methods.

### CROSS-PATENTS REFERENCE

This patent claims the benefit of the U.S. Provisional Patent of Application No. 62/640,858, filed Mar. 9, 2018.

### CONTEXT

Alzheimer's Disease (‘AD’) is a hard-to-tackle neurodegenerative disease observed in elderly population. AD patients develop dementias, especially memory dysfunction, in a progressive and irreversible manner. AD is characterized with the formation of irregular amyloid plaques, and neurofibrillary tangles in brain, which can be found via brain imaging and post mortem dissection. (Wang J. 2017, et al.; Hane F. T., et al., 2017) About each one in ten people of 65 years or older eventually develop Alzheimer's dementia. (Alzheimer's Association, 2017)

In the past few decades, the academia had pushed far on AD's molecular pathogenesis, especially amyloid plaques and tau tangles pathology. (Hane F. T., et al., 2017) In the area of familial AD, researches made strong progressions in the identification of key genetic risk factors, such as APOE genes. However, it is important to note that controversies and mysteries still exist in AD pathology, and AD is yet unpreventable and incurable. All remedial treatments had been announced statistically ineffective in terms of curability or postponement against large population (to name a few: anti-depressants, intranasal insulin, cholinesterase inhibitors, Amyloid Beta Vaccines, Passive immunotherapies, or recently, Dr. Lilly's solanezumab). (Park A., 2016)

Our theory behind such ineffectiveness is that it will be too difficult for medicines that targets only several molecular pathways to rehabilitate everything when neurotoxic already aggregates massively in brain. The key rests in early detection and screening. The earlier the prognosis of AD is, the better chance for the patients. Current implemented screening and early-diagnostic tools are majorly physician-mediated cognitive tests, which could be inconsistent and not sensitive enough for preclinical cognitive impairment detection; in addition, it targets not specifically for Alzheimer's Disease.

Meanwhile, new methods had been developed overtime in the endeavor of early-diagnosis or prediction of neurocognitive disease. Many research groups are working on identifying applicable biomarkers, but those methods are all either too invasive and expensive and “impractical for the early diagnosis of patients without obvious cognitive complaints” (Wang J., et al., 2017). The development of computer-mediated reality had started a new thought. By combining the strong immersive environment with cognitive assessment in order to identify the very mild symptoms before observable biochemical changes (which is often treated as an coordination as well as cloud data services will be needed in cases of extended (or outside-laboratory) device usage for more accurate and complete data and analysis. Certain hardware will be needed to carry out large algorithms or data collection as well.

4. In reference to the methods presented in claim 10-11: Physical feedbacks are implemented in the present invention as alternative ways of giving stimuli, and often those physical stimuli are felt more direct and thus contributes to cognitive function, such as memory formation. This also serves as a tool for claim 64-71.

5. In reference to the methods presented in claim 3-19: Studies had proven that Alzheimer's Disease and many other neurodegenerative diseases affect multiple brain functions, including senses, processing, and retrieval or formation of memory, as a result of cellular dysfunctions or necrosis in the damaged area. For example, the Alzheimer's disease is now theorized to start around the hippocampus, which is manifested as memory dysfunction, then spread over to multiple functional areas. Therefore, appropriate tools shall be implemented to measure the user's performance in those cognitive activities in order to reveal the molecular pathological changes of both general and individual cases.^{1,7}

6. In reference to the methods presented in claim 14-19: most of the apparatuses or neuropsychic tests mentioned in this section are already commonly used in the diagnosis or prognosis of neurodegenerative diseases. Using those methods would help to compare the statistical inferences/significance of collected results and are thus an important part of the present method.

7. In reference to the method presented in claim 21: Cognitive function is divided in this way in order to correspond to the actual tissue specifications of human brain, and function damages are specific to different neurodegenerative diseases as well as different stages. For instance, Alzheimer's Disease also is known for damaging spatial sense and short-term memory, but not long-term memory until in very late stages. Therefore, categorized tests offer more statistically convincing evidences to our analysis.

8. In reference to the methods presented in claim 22-24: Those listed cognitive functions are known to be vulnerable to neurodegenerative diseases; thus, measuring and analyzing potential symptoms or damage of those cognitive functions is the key to evaluations to the present invention. In addition, these cognitive functions listed here will likely to be addressed specifically in our actual product.

9. In reference to the method presented in claim 25: Accompanying events that correlates to cognitive events helps to identify the most prominent symptoms of individual cases, and to formularize the general trend of symptom exposure.

10. In reference to the methods presented in claim 26 and claim 41: Augmented reality, because it does not hinder the user's normal activities as virtual reality does, can execute both observational studies and active experimentations. That is, the device carrying said methods may both prompt users to certain tasks or record data unnoticeably while the users continuing daily routine. Such feature of the augmented reality offers many possible variations in test types and thus better and more holistic analysis of cognitive functions.

11. In reference to the method presented in claim 26: the procedures enumerated here constitutes said active experimentation portions of the method.

12. In reference to the method presented in claim 27, the type of contents of said instructions, signals, tests, interface, or interactions listed here correspond to aforementioned various types of cognitive functions that are vulnerable to neurodegenerative diseases.

13. In reference to the method presented in claim 28: hereby presented are common computer-generated components used in the programming/construction of augmented reality.

14. In reference to the methods presented in claim 29-38: hereby presented are strategies or elements used widely to deliver the necessary components to the evaluation of corresponding cognitive functions.

15. In reference to the method presented in claim 39: it is intuitive to combine the cognitive evaluative function with medical application to test either one's efficacy.

16. In reference to the method presented in claim 40: the present invention offers a new assessment method for cognitive function. It is believed that (CITATION) cognitive function should improve in cases when external stimuli are present. For example, it is known that humans form deeper and longer memories about intense experience or when are told to actively memorize, such as a fight-or-flight experience or to promptly memorize formula for a math test. The present invention takes such feature of human cognition and identify underperformance when said external stimuli favorable to better cognitive function are present as a signal to potential cognitive dysfunction.

17. In reference to the method presented in claim 41: further, the enumerated quantities or qualities are likely to be addressed specifically in the invention.

18. In reference to the methods presented in claim 42-44: further, the enumerated types of illnesses here are likely to be the sources of cognitive underperformance found by aforementioned methods.

19. In reference to the method presented in claim 45-47: the present method is most likely to be constructed with Software, such as Unity engine as well as other digital tools. The enumerated Software components in this present invention is the most intuitive way to program and execute said Augmented Reality.

20. In reference to the method presented in claim 48-51: the present method addresses the use of the data produced in the method of claim 1. Information such as overall cognitive ratings or long-term cognitive performance are valuable data for, for instance, the primary diagnosis of Alzheimer's Disease. Specific types that are major interest of the present invention are also enumerated.

21. In reference to the method presented in claim 52: Further, as section 15 of the description said, the method can be further used to validify the efficacy of other treatments.

22. In reference to the method presented in claim 53: Further, specific therapeutic interventions that are the most intuitive options are listed.

23. In reference to the method presented in claim 54: APOE ϵ3, APOE ϵ2, APOE ϵ4 are three allele that have been identified to associate with Alzheimer's Disease's pathology, especially early-onset types. The present invention further addressed the use of genetic studies to enhance or validify analysis and related medical treatments, as well as draw conclusion from population data generated by aforementioned methods. Specific types of medical treatments are most common in treatment of certain aforementioned sources of cognitive dysfunctions (e.g. Alzheimer's Disease or Parkinson's disease) are enumerated.¹

24. In reference to the methods presented in claim 55 and claim 59: Further, method of claim 55 further addressed the use of long-term results and corresponding statistical and experimental approaches. It is understood that most sources of cognitive dysfunctions show subtle changes that are only evident with large quantities of data. Also, individual background differences greatly influence data generated by aforementioned methods. The present method addresses this issue by comparing one's dataset with both themselves and other individuals for more systematic and accurate analysis.

25. In reference to the method presented in claim 56: The present method addresses the common strategy of comparing users with healthy individuals and high-risk population to facilitate analysis.

26. In reference to the methods presented in claim 57-58: The present methods address selecting specific high Alzheimer's Diseases risk population as the aforementioned comparison. As explained before, APOE is one of the commonly accepted indicators of risk to Alzheimer's Disease.

27. In reference to the methods presented in claim 60-62: it will be more favorable to both the users and the market when the method is completed by an integrated system.

28. In reference to the method presented in claim 63: The “segments” refer to time-intervals that users occupy to interact with systems that involve aforementioned methods. This method addresses the option of taking the advantage of Augmented Reality's functionality of conducting data collection and processing in multiple time intervals, meanwhile leaving user's normal daily routine unhindered.

29. In reference to the method presented in claim 64: Augmented Reality, besides being able to screen cognitive dysfunctions said methods before, also serves as a way to improve cognitive function as it offers intense and immersive environment. As explained in section 16 of the Description, improvement of cognitive function can also be achieved by presenting said external stimuli in the Augmented Reality environment.

30. In reference to the methods presented in claim 65, claim 66, and claim 67: these two methods address some specific aspects that the medical community has known to be crucial for cognitive functions, and the corresponding test methods.

31. In reference to the method presented in claim 68, this method addresses the option of reiterate the assessment process to determine the device's efficacy in cognitive improvement. Then in reference to the method presented in claim 69, the method from previous claims are specifically chosen.

32. In reference to the method presented in claim 70, this is a specific routine that can be implemented.

33. In reference to the method presented in claim 71, this method addresses specific pathological improvements that are widely referred in the medical community.

34. In reference to the method presented in claim 72, this method addresses a specific disease and its symptom that the claimed methods can be utilized on.

### ACKNOWLEDGEMENTS

1. Wang, J., Gu, B. J., Masters, C. L., & Wang, Y. (2017). A systemic view of Alzheimer disease—insights from amyloid-β metabolism beyond the brain. Nature Reviews Neurology, 13(11), 703-703. doi:10.1038/nmeurol.2017.147
2. Hane, F. T., Lee, B. Y., & Leonenko, Z. (2017). Recent Progress in Alzheimer's Disease Research, Part 1: Pathology. Journal of Alzheimers Disease, 57(1), 1-28. doi:10.3233/j ad-160882
3. Hane, F. T., Robinson, M., Lee, B. Y., Bai, O., Leonenko, Z., & Albert, M. S. (2017). Recent Progress in Alzheimer's Disease Research, Part 3: Diagnosis and Treatment. Journal of Alzheimer's Disease, 57(3), 645-665. http ://doi.org/10.3233/JAD-160907
4. 2017 Alzheimer's Disease Facts and Figures [PDF]. (2017). Alzheimer's Association
5. Park, A. (2016, November 23). Eli Lilly Alzheimer's Disease Drug Fails at Trial. Retrieved March 10, 2018, from http://time.com/4581267/eli-lilly-company-alzheimers-disease-drug
6. Virtual Reality Vs. Augmented Reality Vs. Mixed Reality. (n.d.). Retrieved from https://www.intel.com/content/www/us/en/tech-tips-and-tricks/virtual-reality-vs-augmented-reality.html
7. Portet, F., Ousset, P. J., Visser, P. J., Frisoni, G. B., Nobili, F., Scheltens, P., Vellas, B., Touchon, J., MCI Working Group of the European Consortium on Alzheimer's Disease (EADC) (2006). Mild cognitive impairment (MCI) in medical practice: a critical review of the concept and new diagnostic procedure. Report of the MCI Working Group of the European Consortium on Alzheimer's Disease. Journal of neurology, neurosurgery, and psychiatry, 77(6), 714-8.
8. Carnoy, D. (2018, March 09). Bose is making AR glasses. Retrieved from https://www.cnet.com/news/bose-yes-that-bose-will-develop-augmented-reality-glasses/

What is claimed is:

1. A method, comprising:
a. user's interaction with said Augmented Reality, or a variation based on Mixed Reality.
b. Measurement or evaluation of user's performance in reality or the Computer-generated or Computer-modified environment;
c. Analysis or evaluation of user's cognitive performance, status, or potential treatments.

2. The method of claim 1, wherein the user's experience or perception of the real-world is subject to one or more of the following: visual, auditory, haptic, somatosensory, olfactory, or gustatory.

3. The method of claim 1, wherein the operation of the present invention further includes one or more of the following:
a. the use of hardware, sensors.
b. the use or coordinate systems, data generation, data collection, data processing, or analysis.
c. the use of clinical procedures.

4. The method of claim 3, wherein the hardware or sensor includes one or more of the following: computer, servers, smart devices, input devices, interfaces, data collecting devices.

5. The method of claim 4, wherein the input devices include one or more of the following: touch-based devices, gesture-based devices, posture-based devises, motion-based devices, remote, controller, pen akin devices, keyboards, and mouse.

6. The method of claim 4, wherein the interface is a visual interface.

7. The method of claim 6, wherein the visual interface includes either the use of an image-presenting-goggles or projector.

8. The method of claim 4, wherein the interface is an auditory interface.

9. The method of claim 8, wherein the auditory interface includes the use of a speaker.

10. The method of claim 4, wherein the interface is based on physical feedbacks.

11. The method of claim 10, wherein the physical feedbacks is one or more of the following methods: vibration, electrical stimuli, or scent generation.

12. The method of claim 4, wherein the data collecting device includes one or more of the following: motion tracking devices, camera, or microphone.

13. The method of claim 12, wherein the motion tracking devices include one or more of the following: motion sensors, motion trackers, and view trackers.

14. The method of claim 3, wherein the clinical procedures is a physical examination, clinical assessment, or therapeutic intervention.

15. The method of claim 14, wherein the physical examination is an imaging procedure.

16. The method of claim 15, wherein the imaging procedure is one or more of the following: Magnetic Resonance Imaging, Electroencephalogram, Computer Tomography, Positron emission tomography, Infrared Imaging.

17. The method of claim 14, wherein the physical examination involves genetic testing, cerebrospinal fluid, thermometer, or Pulse sensor.

18. The method of claim 14, wherein the clinical assessment is a clinical inquiry.

19. The method of claim 18, wherein the clinical inquiry is a cognitive assessment or psychological assessment.

20. The method of claim 1, wherein the cognitive status or performance includes one or more or the combination of the following: cognitive function or emotions.

21. The method of claim 20, wherein the cognitive function includes one or more of the following: episodic memory, long-term memory, short-term memory, visual memory, auditory memory, tactile memory, spatial memory, language process and memory, abstract function and memory.

22. The method of claim 21, wherein the spatial memory includes one or more of the following: three-dimensional memory, two-dimensional memory, or abstract spatial memory.

23. The method of claim 21, wherein the abstract function and memory is referring the function or memory of one or more of the following: numbers, algebra, arithmetic, logics, reasoning, or abstract ideas.

24. The method of claim 20, wherein the cognitive status of emotions includes one or more of the following: shift in personalities, patterns of emotion, or emotional stability.

25. The method of claim 24, where in the cognitive status of emotions is correlated with events.

26. The method of claim 1, wherein the process of measuring performance or evaluation involves giving instructions, signals, tests, interface, or interaction that contribute to the analysis or assessment of the subject's cognitive status or performance.

27. The method of claim 26, wherein the process of giving instructions, signals, testing, interfacing, or interacting involves presenting or applying one or more of the following: visual information and experience, auditory information and experience, tactile information and experience, spatial scenery and information, language information and experiences, abstract information.

28. The method of claim 27, wherein the presentation or application of visual information and experience involves the presentation of one or more of the following: messages, signs, geometries, shapes, bodies, images, colors, shades, models, special effects, and sceneries, either animated or still.

29. The method of claim 27, wherein the presentation or application of auditory information and experience involves the presentation of sounds or rhythms.

30. The method of claim 27, wherein the presentation or application of tactile information and experience involves the presenting the users tactile sensations.

31. The method of claim 27, wherein the presentation or application of spatial scenery and information involves augmenting or presenting three-dimensional or two-dimensional scenery.

32. The method of claim 31, wherein the three-dimensional or two-dimensional scenery includes maze or street view.

33. The method of claim 27, wherein the presentation or application of language information and experience involves the presentation of one or more of the characters, words, texts, sounds of natural, artificial, or transformed languages.

34. The method of claim 33, wherein the transformation is applied to a language's visual, tactile, or auditory attributes.

35. The method of claim 33, wherein the transformation is achieved by either transforming forms, sizes, fonts, or pronunciations.

36. The method of claim 33, wherein the presentation of either characters, words, texts, sounds is built after a specific person, genre, or style.

37. The method of claim 36, wherein the specific person is an acquaintance of the user.

38. The method of claim 27, wherein the presentation or application of abstract information involves the presentation of one or more of the following: questions related to numbers, arithmetic, reasoning, or logics.

39. The method of claim 14, wherein therapeutic intervention involves the use of either drug, neuropsychic stimuli, or a treatment plan.

40. The method of claim 26, wherein the test, interface, or interaction involves one or more of the following:
a. Presenting a simulation or description of a virtual or real event;
b. Assessing user's memory of experience with a virtual or real event;
c. Impact user's measured performance when he fails to identify the validity, frequency, or contents of the event.

41. The method of claim 1, wherein the measured performance includes one or more of the following: distance traversed (either in the reality or in the augmented portion), velocity of the user or entities, time elapsed for completing a task or event, space-time information about an event, situational information about an event, correlating data with a difficulty or complexity indication, completion rates, successful rates, quantity of completed or successful tasks or events occurred, qualification of completed or successful tasks and events occurred, descriptive or categorical data about the surroundings or the users, body or body parts position information, time-stamped data, data about user's attention, user's feedback, neuropsychic activities, pattern of user's reaction or input.

42. The method of claim 1, wherein the cognitive status is subject to a neuropsychic or psychological disease.

43. The method of claim 42, wherein the neuropsychic disease is a neurodegenerative disease.

44. The method of claim 43, wherein the neurodegenerative disease is Alzheimer's Disease, Early-onset Alzheimer's Disease, or Attention Deficit Hyperactivity Disorder.

45. The method of claim 1, wherein the Augmented Reality includes the use of software for coordination, execution, or measurement of performance, or the collection or analysis of data, or the analysis of cognitive performance and status.

46. The method of claim 45, further comprising having the virtual entity's spatial attributes being coordinated by vector data.

47. The method of claim 45, further comprising having the virtual entity's temporal attributes being coordinated by the Software.

48. The method of claim 1, wherein the analysis of cognitive status or performance is used in the diagnosis of, the prognosis of, the evaluation of, or identification of the risk/chance of acquiring: cognitive decline, cognitive improvement, or cognitive disability.

49. The method of claim 48, wherein the cognitive decline is subject to Alzheimer's disease.

50. The method of claim 48, wherein the cognitive improvement involves one or more of the following: memory improvement, learning performance improvement, and emotional stability.

51. The method of claim 48, wherein the cognitive disability is either: Attention-deficit/hyperactivity disorder, memory performance problem, learning performance problem, or early onset Alzheimer's Disease.

52. The method of claim 1, wherein the analysis of cognitive status or the measurement of performance is used to evaluate the performance of a therapeutic intervention or proposed one.

53. The method of claim 52, wherein the therapeutic intervention includes one or more of the following: Drugs, Stimuli, or Change of life style.

54. The method of claim 53 or the method of claim 39, wherein the drug is an APOE ϵ3, APOE ϵ2, APOE ϵ4 mimetic, a cholinesterase inhibitor, an N-methyl-aspartate receptor antagonist, a hormone therapy, a vitamin, or cannabinoid.

55. The method of claim 1, wherein the process of analysis involves one or more of the following:
a. Comparing the user's input, measured performance, and data collected with oneself;
b. Comparing user's input, measured performance, or data collected with other subjects;
c. Measuring or analyzing the pattern of events, event types, or event frequencies;
d. Or, analyzing the user's adaptation to the method.

56. The method of claim 55, wherein the other subjects in “the comparison of user's input Comparing user's input, measured performance, or data collected with other subjects” are subject to cognitive impairment, disability or have high risk in developing one.

57. The method of claim 56, wherein the cognitive impairment or the risk of development therefore is subject to Alzheimer's Disease.

58. The method of claim 56, wherein the other subjects are subject APOE genetic risk.

59. The method of claim 55, wherein the comparison is drawn to eliminate variation caused by age, gender, or handicap.

60. The method of claim 1, further having its elements being achieved or performed by an integrated system.

61. The method of claim 61, wherein the integrated system is an integrated software.

62. The method of claim 61, wherein the integrated system is an integrated hardware.

63. The method of claim 1, wherein the interaction compromise segments of interaction.

64. The method of claim 1, further compromising: presenting program or contents that renders a stimulating experience in the augmented reality along with certain events or tasks.

65. The method of claim 64, wherein the program or contents' stimulating experience is achieved by one or more of the following:
a. presentation of warning visual cues, visual effects, interface, or messages;
b. presentation of warning sounds, sound effects, or auditory messages;
c. presentation of a tactile sensations, or a pattern of one;
d. presentation of a stimulating scenery, entity, or environment in the augmented reality.

66. The method of claim 64, wherein the instructions or tasks is related to the cognitive functions or status in one or more of the following ways:
a. it prompts or assesses the user's episodic memory ability;
b. it prompts or assesses the user's spatial memory;
c. it prompts or assesses the user's short-term memory;
d. it prompts or assesses the user's sensory memory;
e. it prompts or assesses the user's comprehensive functions;
f. it prompts or assesses the user's emotional stabilities.

67. The method of claim 66, wherein the comprehensive function is the ability to process or memorize: languages, abstract ideas, numbers, or algebra.

68. The method of claim 64, further comprises the assessment of the effects of the cognitive status or function improvement.

69. The method of claim 68, further comprising: integrate the result of the assessment in measuring cognitive risk, cognitive status analysis, or method of claim 1.

70. The method of claim 69, wherein the integration is as follows: if the user has unsatisfactory improvement, the user's cognitive status or function rating decrease.

71. The method of claim 64, wherein the improvement of cognitive status or function is referring to the mitigation, cure, postponement of a neuropsychic or neurodegenerative disease.

72. The method of claim 71, wherein the neurodegenerative disease is Alzheimer's Disease or its early-onset/familial variants.
